(19) United States
(12) Patent Application Publication
(10) Pub. No.: US 2025/0186026 A1
(43) Pub. Date: Jun. 12, 2025

(54) Ultrasound Probe with Pressure Measurement Capability

(71) Applicant: Bard Access Systems, Inc., Salt Lake City, UT (US)

(72) Inventor: Huy Ngoc Tran, Riverton, UT (US)

(21) Appl. No.: 19/058,331

(22) Filed: Feb. 20, 2025

Related U.S. Application Data
(63) Continuation of application No. 17/471,015, filed on Sep. 9, 2021, now Pat. No. 12,232,910.
(60) Provisional application No. 63/076,589, filed on Sep. 10, 2020.

(51) Int. Cl. A61B 8/00; A61B 8/08

(52) U.S. Cl. CPC A61B 8/4444; A61B 8/0841; A61B 8/085; A61B 8/463

(57) ABSTRACT

Ultrasound probes, ultrasound systems, and ultrasound methods with pressure measurement capabilities for detecting and determining if bodily tissue is over-compressed during ultrasound imaging procedure. An ultrasound probe can include a probe body, an articulating probe head attached to the probe body, and a pressure-sensing device housed in an articulating area between the articulating probe head and the probe body. A method can include placing the articulating probe head of the ultrasound probe on a skin surface of a patient and moving the articulating probe head of the ultrasound probe over the patient while ultrasound signals are emitted into the patient from the articulating probe head. The method can also include monitoring for measured pressure values induced on the patient by the articulating probe head to determine whether a threshold pressure value has been exceeded.

### PRIORITY

This application is a continuation of U.S. patent application Ser. No. 17/471,015, filed Sep. 9, 2021, now U.S. Pat. No. 12,232,910, which claims the benefit of priority to U.S. Provisional Application No. 63/076,589, filed Sep. 10, 2020, each of which is incorporated by reference in its entirety into this application.

### BACKGROUND

There are currently a variety of existing ultrasound systems including wired or wireless ultrasound probes that connect to displays. These systems can be used by clinicians for assessing a site such as a blood vessel for placing a vascular access device (“VAD”) including a catheter. These systems can also by clinicians for assessing placement of the VAD or catheter at a chosen site. However, bodily tissue of a patient can be appreciably compressed while assessing such sites by simply using the ultrasound probes as intended. Compression of the bodily tissue can compromise vessel purchase by the VAD or catheter, which, in turn, can result in catheter extravasation that can be dangerous to the patient's health. Existing ultrasound systems do not provide for measuring bodily tissue-compressing pressure caused by the ultrasound probes during ultrasound imaging.

Disclosed herein are ultrasound probes, ultrasound systems, and ultrasound methods with pressure measurement capabilities for detecting and determining if bodily tissue is over-compressed during ultrasound imaging.

### SUMMARY

Disclosed herein is an ultrasound probe including, in some embodiments, a probe body, an articulating probe head attached to the probe body, and a pressure-sensing device housed in an articulating area between the articulating probe head and the probe body.

In some embodiments, the ultrasound probe further includes a boot connecting the articulating probe head to the probe body in the articulating area. The boot is configured to cover or incorporate therein the pressure-sensing device.

In some embodiments, the pressure-sensing device is configured to detect deformations in or around an elastic material of the boot. The deformations are induced by pressing the articulating probe head into a patient.

In some embodiments, the pressure-sensing device is communicatively coupled to a controller of the ultrasound probe. The controller is configured to convert electrical signals corresponding to the deformations into measured pressure values.

In some embodiments, the ultrasound probe is configured to provide the measured pressure values to a display to be displayed to a clinician.

In some embodiments, the ultrasound probe includes logic configured to compare a measured pressure value against a threshold pressure value.

In some embodiments, the ultrasound probe includes a speaker configured to emit an audio signal to alert a clinician when the measured pressure value exceeds the threshold pressure value.

In some embodiments, the ultrasound probe includes a light-emitting diode configured to emit a visual signal to alert a clinician when the measured pressure value exceeds the threshold pressure value.

In some embodiments, the pressure-sensing device is a pressure transducer.

In some embodiments, the pressure transducer is a piezoresistive strain-gauge pressure transducer. The pressure transducer includes a strain gauge bonded to a flexible diaphragm in the articulating area between the articulating probe head and the probe body. A deformation in the diaphragm provides a corresponding measurable change in strain-gauge electrical resistance indicative of the pressure induced by pressing the articulating probe head into a patient to cause the deformation.

In some embodiments, the pressure transducer is a variable capacitance pressure transducer. The pressure transducer includes a diaphragm electrode and an opposing electrode in the articulating area between the articulating probe head and the probe body. A deformation in a flexible diaphragm affects a distance between the diaphragm electrode and the opposing electrode providing a corresponding measurable change in capacitance indicative of the pressure induced by pressing the articulating probe head into a patient to cause the deformation.

Also disclosed herein is an ultrasound system including, in some embodiments, a console and an ultrasound probe. The console includes a display configured for rendering ultrasound images on a display screen of the display. The ultrasound probe includes a probe body, an articulating probe head attached to the probe body, and a pressure-sensing device housed in an articulating area between the articulating probe head and the probe body.

In some embodiments, the ultrasound probe further includes a boot connecting the articulating probe head to the probe body in the articulating area. The boot is configured to cover or incorporate therein the pressure-sensing device.

In some embodiments, the pressure-sensing device is configured to detect deformations in or around an elastic material of the boot. The deformations are induced by pressing the articulating probe head into a patient.

In some embodiments, the pressure-sensing device is communicatively coupled to a controller of the console. The controller is configured to convert electrical signals corresponding to the deformations into measured pressure values.

In some embodiments, the ultrasound probe is configured to provide the measured pressure values to the display to be displayed to a clinician.

In some embodiments, the console includes logic configured to compare a measured pressure value against a threshold pressure value.

In some embodiments, the console includes a speaker configured to emit an audio signal to alert a clinician when the measured pressure value exceeds the threshold pressure value.

In some embodiments, the display is configured to emit a visual signal to alert a clinician when the measured pressure value exceeds the threshold pressure value.

In some embodiments, the display is configured to display visual feedback including a visualization of a target vein and a catheter placed in the target vein.

In some embodiments, the pressure-sensing device is a piezoresistive strain-gauge pressure transducer. The pressure transducer includes a strain gauge bonded to a flexible diaphragm in the articulating area between the articulating probe head and the probe body. A deformation in the diaphragm provides a corresponding measurable change in strain-gauge electrical resistance indicative of the pressure induced by pressing the articulating probe head into a patient to cause the deformation.

In some embodiments, the pressure-sensing device is a variable capacitance pressure transducer. The pressure transducer includes a diaphragm electrode and an opposing electrode in the articulating area between the articulating probe head and the probe body. A deformation in a flexible diaphragm affecting a distance between the diaphragm electrode and the opposing electrode providing a corresponding measurable change in capacitance indicative of the pressure induced by pressing the articulating probe head into a patient to cause the deformation.

Also disclosed herein is a method of an ultrasound system including, in some embodiments, an ultrasound probe-obtaining step, an ultrasound probe-placing step, an ultrasound probe-moving step, and a pressure-monitoring step. The ultrasound probe-obtaining step includes obtaining the ultrasound probe. The ultrasound probe includes a probe body, an articulating probe head attached to the probe body, and a pressure-sensing device housed in an articulating area between the articulating probe head and the probe body. The ultrasound probe-placing step includes placing the articulating probe head of the ultrasound probe on a skin surface of a patient. The ultrasound probe-moving step includes moving the articulating probe head of the ultrasound probe over the patient while ultrasound signals are emitted into the patient from the articulating probe head for ultrasound imaging. The pressure-monitoring step includes monitoring for any measured pressure values induced on the patient by the articulating probe head of the ultrasound probe in excess of a threshold pressure value.

In some embodiments, the pressure-monitoring step includes viewing the measured pressure values on a display screen of a display.

In some embodiments, the pressure-monitoring step includes monitoring for a visual signal on the display screen of the display that alerts a clinician when any measured pressure values are in excess of the threshold pressure value.

In some embodiments, the pressure-monitoring step includes monitoring for an audio signal that alerts a clinician when any measured pressure values are in excess of the threshold pressure value.

In some embodiments, the method further includes a catheter placement-adjusting step. The catheter placement-adjusting step includes adjusting catheter placement responsive to any measured pressure values in excess of the threshold pressure value to ensure a sufficient blood-vessel purchase that minimizes catheter extravasation.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a wired ultrasound system including a console and an ultrasound probe in accordance with some embodiments.

FIG. 2 illustrates a wireless ultrasound system including a console and an ultrasound probe in accordance with some embodiments.

FIG. 3 illustrates a block diagram of the ultrasound system of FIG. 1 in accordance with some embodiments.

FIG. 4 illustrates a wired ultrasound system including a companion device and an ultrasound probe in accordance with some embodiments.

FIG. 5 illustrates a wireless ultrasound system including a companion device and an ultrasound probe in accordance with some embodiments.

FIG. 6 illustrates a block diagram of the ultrasound system of FIG. 2, FIG. 4, or 5 in accordance with some embodiments.

FIG. 7 illustrates a front view of an ultrasound probe including a pressure-sensing device in accordance with some embodiments.

FIG. 8 illustrates a perspective view of the ultrasound probe in accordance with some embodiments.

FIG. 9 illustrates a cross section of the ultrasound probe in accordance with some embodiments.

FIG. 10 illustrates another cross section of the ultrasound probe in accordance with some embodiments.

FIG. 11 illustrates a detailed view of an articulating area of the ultrasound probe including the pressure-sensing device in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Lastly, in the following description, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

Unless defined otherwise, all technical and scientific terms used herein have the same meaning as commonly understood by those of ordinary skill in the art.

As set forth above, existing ultrasound systems do not provide for measuring bodily tissue-compressing pressure caused by ultrasound probes during ultrasound imaging. Disclosed herein are ultrasound probes, ultrasound systems, and ultrasound methods with pressure measurement capabilities for detecting and determining if bodily tissue is over-compressed during ultrasound imaging.

### Ultrasound Systems

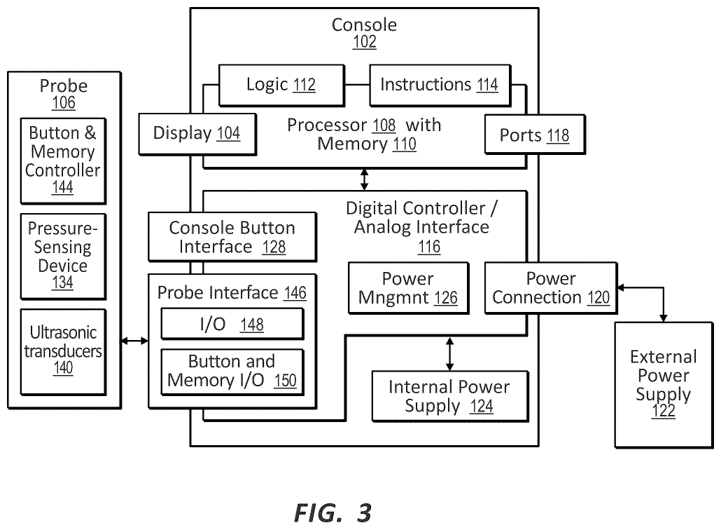

FIG. 1 illustrates a wired ultrasound system 100 in accordance with some embodiments. FIG. 3 illustrates a block diagram of the wired ultrasound system 100 in accordance with some embodiments.

As shown, the wired ultrasound system 100 includes a console 102, a display 104, and a wired ultrasound probe 106. During operation of the wired ultrasound system 100, the articulating probe head 132 of the wired ultrasound probe 106 is placed against skin of a patient. An ultrasound beam is produced so as to ultrasonically image a portion of a target such as a blood vessel beneath a surface of the skin of the patient. The ultrasonic image of the blood vessel can be depicted on the display screen of the display 104 along with the measured pressure values as set forth below. The wired ultrasound system 100 is useful for assessing access sites such as assessing a blood vessel within a body of a patient before making a percutaneous puncture with a needle to place a VAD such as a catheter into the blood vessel. The wired ultrasound system 100 is also useful for assessing access sites subsequent to placing VADs. However, it should be appreciated that the wired ultrasound system 100 can be useful in a variety of ultrasound-based medical procedures other than catheterization. For example, the percutaneous puncture with the needle can be performed to biopsy tissue of an organ of the patient.

The console 102 houses a variety of components of the wired ultrasound system 100, and it is appreciated the console 102 can take any of a variety of forms. A processor 108 and memory 110 such as random-access memory (“RAM”) or non-volatile memory (e.g., electrically erasable programmable read-only memory [“EEPROM”]) is included in the console 102 for controlling various functions of the wired ultrasound system 100, as well as executing various logic operations or algorithms via logic 112 during operation of the wired ultrasound system 100 in accordance with executable instructions 114 therefor stored in the memory 110 for execution by the processor 108. For example, the console 102 is configured to instantiate by way of the instructions 114 one or more processes for controlling the functions of the wired ultrasound system 100, processing electrical signals from the ultrasonic transducers 140 of the wired ultrasound probe 106 into ultrasound images, processing electrical signals from the pressure-sensing device of the wired ultrasound probe 106 into measured pressure values, etc. A digital controller/analog interface 116 is also included with the console 102 and is in communication with both the processor 108 and other system components to govern interfacing between the wired ultrasound probe 106 and other system components set forth herein.

A controller of the console 102, optionally implemented between the processor 108 and the memory 110 of the console 102, is communicatively coupled to the pressure-sensing device 134 of the wired ultrasound probe 106 set forth below. The controller is configured to convert electrical signals corresponding to deformations of the boot 138 of the wired ultrasound probe 106 into measured pressure values, the deformations being those in or around the elastic material of the boot 138 induced by pressing the articulating probe head 132 into a patient. Notably, the logic 112 of the console 102 is configured to compare each measured pressure value against a threshold pressure value to alert a clinician when a measured pressure value exceeds the threshold pressure value. For example, the console 102 can include a speaker configured to emit an audio signal to alert the clinician when the measured pressure value exceeds the threshold pressure value. In another example, the display 104 is configured to emit a visual signal on the display screen to alert a clinician when the measured pressure value exceeds the threshold pressure value.

The wired ultrasound system 100 further includes ports 118 for connection with additional components such as optional components including a printer, storage media, a keyboard, etc. The ports 118 can be universal serial bus (“USB”) ports, though other types of ports can be used for this connection or any other connections shown or described herein.

A power connection 120 is included with the console 102 to enable an operable connection to an external power supply 122. An internal power supply 124 (e.g., a battery) can also be employed either with or exclusive of the external power supply 122. Power management circuitry 126 is included with the digital controller/analog interface 116 of the console 102 to regulate power use and distribution.

The display 104 includes a display screen integrated into the console 102 to provide a graphical user interface (“GUI”), render one or more ultrasound images of the target (e.g., the blood vessel) attained by the wired ultrasound probe 106, and display 104 any related information such as the measured pressure values for the articulating probe head 132 when attaining the one-or-more ultrasound images. In addition, the display 104 can be configured to display visual feedback including a visualization of a target (e.g., a blood vessel such as a vein) and a VAD such as a catheter placed in the target. Notwithstanding the foregoing, the display 104 can alternatively be separate from the console 102 and communicatively coupled thereto. Control buttons (see FIG. 1) accessed through a console button interface 128 of the console 102 can be used to immediately call up a desired mode of the wired ultrasound system 100 to the display screen for assistance in an ultrasound-based medical procedure such as assessing the foregoing target or placing a VAD therein.

The wired ultrasound probe 106 is employed in connection with ultrasound-based visualization of a target such as a blood vessel in preparation for placing a VAD such as a catheter into the target. Such visualization gives real-time ultrasound guidance and assists in reducing complications commonly associated with VAD placement such as catheter extravasation. The wired ultrasound probe 106 is configured to provide to the console 102 electrical signals from the ultrasonic transducers 140 of the wired ultrasound probe 106, electrical signals from the pressure-sensing device of the wired ultrasound probe 106, or a combination thereof for real-time ultrasound guidance in VAD placement or other medical procedures.

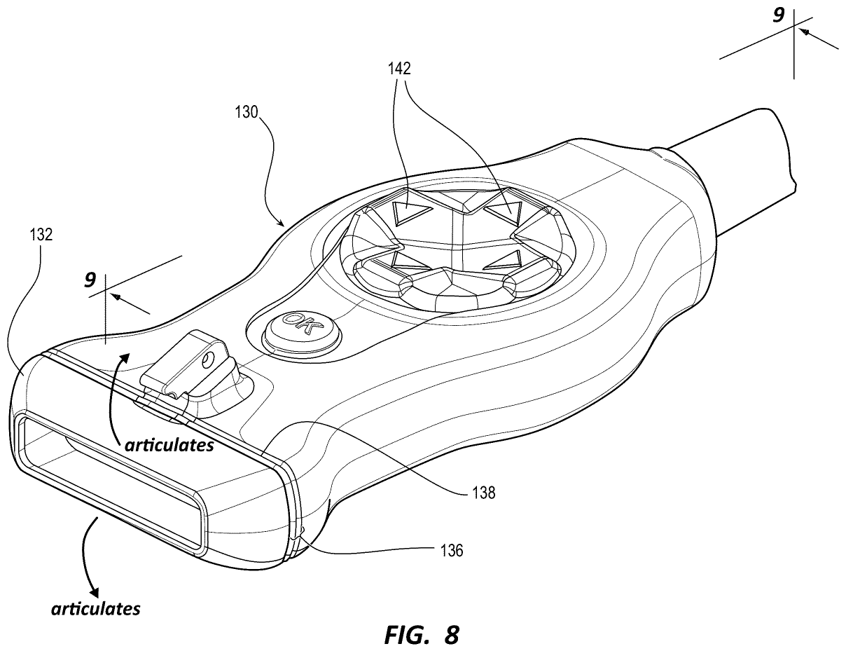

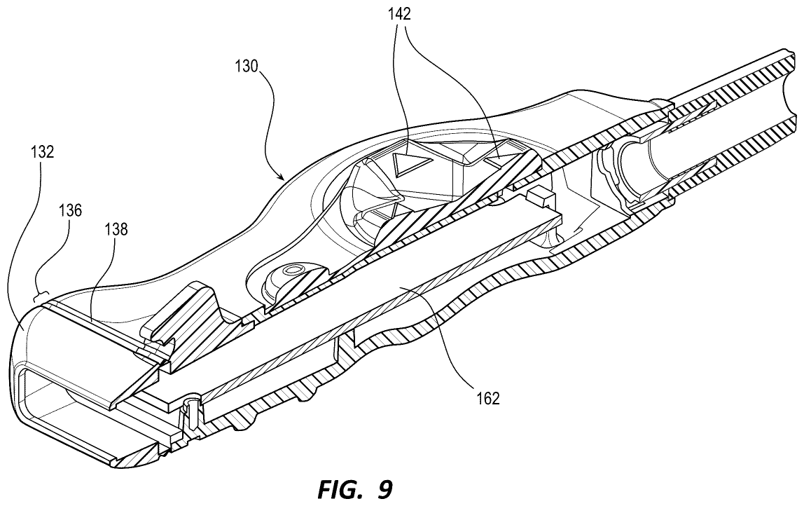

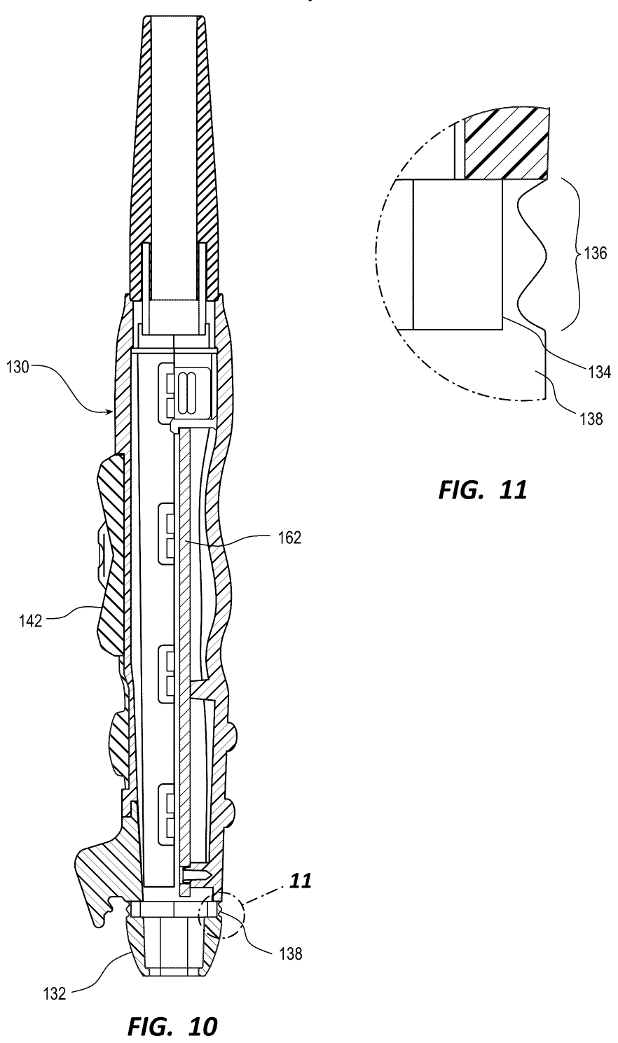

FIGS. 1 and 7-11 illustrate various views of the wired ultrasound probe 106 in accordance with some embodiments.

As shown, the wired ultrasound probe 106 includes a probe body 130, an articulating probe head 132 attached to the probe body 130, and a pressure-sensing device 134 housed in an articulating area 136 between the articulating probe head 132 and the probe body 130. The wired ultrasound probe 106 further includes a boot 138 connecting the articulating probe head 132 to the probe body 130 in the articulating area 136. The boot 138 is configured to cover or incorporate therein the pressure-sensing device 134.

The articulating probe head 132 houses an array of ultrasonic transducers 140, wherein the ultrasonic transducers 140 are piezoelectric ultrasonic transducers or capacitive micromachined ultrasonic transducers (“CMUTs”). The articulating probe head 132 is configured for placement against skin of a patient proximate a prospective VAD placement site where the ultrasonic transducers 140 in the articulating probe head 132 can generate and emit the generated ultrasound signals into the patient in a number of pulses, receive reflected ultrasound signals or ultrasound echoes from the patient by way of reflection of the generated ultrasonic pulses by the body of the patient, and convert the reflected ultrasound signals into corresponding electrical signals for processing into ultrasound images by the console 102. In this way, a clinician can employ the wired ultrasound system 100 to determine a suitable VAD placement site and establish vascular access therewith.

The pressure-sensing device 134 is configured to detect deformations in or around an elastic material of the boot 138, which deformations are induced by pressing the articulating probe head 132 into a patient. The pressure-sensing device 134 can be a pressure transducer or a number of pressure transducers. For example, the pressure transducer can be a piezoresistive strain-gauge pressure transducer. Such a pressure transducer includes a strain gauge bonded to a flexible diaphragm in the articulating area 136 between the articulating probe head 132 and the probe body 130. A deformation in the diaphragm provides a corresponding measurable change in strain-gauge electrical resistance indicative of the pressure induced by pressing the articulating probe head 132 into the patient to cause the deformation. In another example, the pressure transducer is a variable capacitance pressure transducer. Such as pressure transducer includes a diaphragm electrode and an opposing electrode in the articulating area 136 between the articulating probe head 132 and the probe body 130. A deformation in a flexible diaphragm affects a distance between the diaphragm electrode and the opposing electrode providing a corresponding measurable change in capacitance indicative of the pressure induced by pressing the articulating probe head 132 into the patient to cause the deformation.

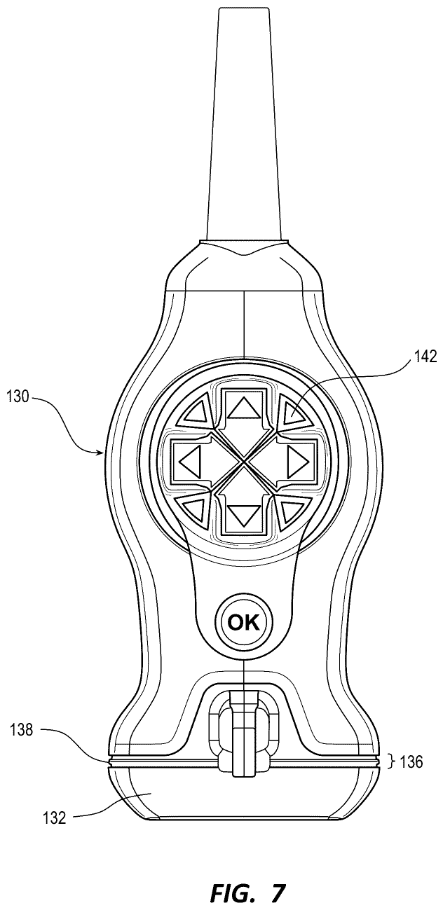

The wired ultrasound probe 106 further includes control buttons 142 for controlling certain aspects of the wired ultrasound system 100 during an ultrasound-based medical procedure, thus eliminating the need for the clinician to reach out of a sterile field around a patient to control the wired ultrasound system 100. For example, the control buttons 142 (see FIG. 7) included on the wired ultrasound probe 106 can be used to immediately call up a desired mode to the display screen by the clinician for assistance in VAD placement or some other an ultrasound-based medical procedure.

FIG. 3 shows that the wired ultrasound probe 106 further includes a button-and-memory controller 144 for governing button and ultrasound-probe operation. The button-and-memory controller 144 can include non-volatile memory (e.g., EEPROM). The button-and-memory controller 144 is in operable communication with a probe interface 146 of the console 102, which includes an input/output (“I/O”) component 148 for interfacing with the ultrasonic transducers 140 and a button and memory I/O component 150 for interfacing with the button-and-memory controller 144.

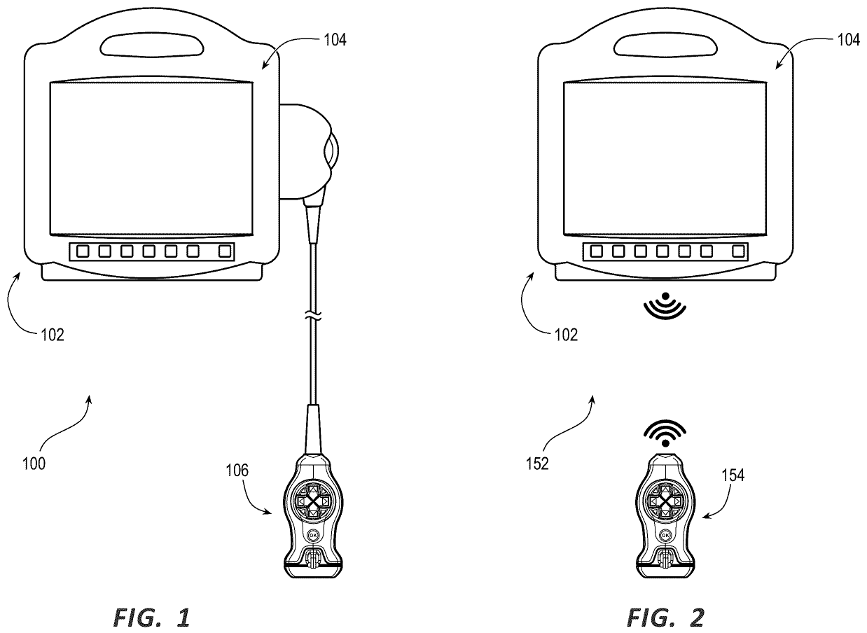

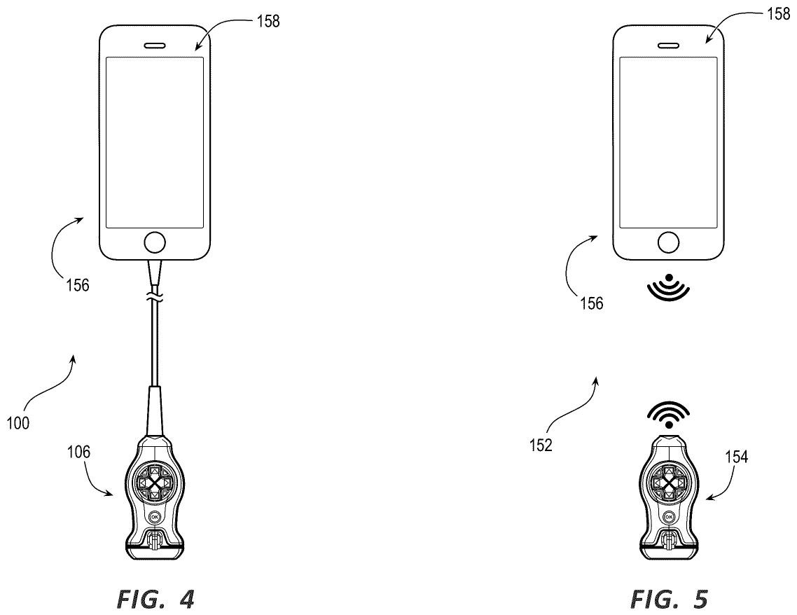

FIGS. 2 and 5 illustrate a wireless ultrasound system 152 in accordance with some embodiments. FIG. 4 illustrates the wired ultrasound system 100 in accordance with some other embodiments than those set forth above; indeed, the wired ultrasound probe 106 shown in FIG. 4 is like the wireless ultrasound probe 154 in that the processing of the electrical signals from the ultrasonic transducers 140 and the pressure-sensing device 134 of the wired ultrasound probe 106 is by the wired ultrasound probe 106, itself, for display on the companion device 156 through a wired connection instead of a wireless connection. FIG. 3 illustrates a block diagram of the wireless ultrasound system 152 in accordance with some embodiments.

While description of the wireless ultrasound system 152 is set forth below, it should be understood that the wireless ultrasound system 152 includes similar components to the wired ultrasound system 100 set forth above, albeit distributed differently about the wireless ultrasound system 152. For example, the wireless ultrasound probe 154, itself, can include the processor 108, the memory 110, the instructions 114, and the logic 112 of the console 102 for controlling various functions of the wireless ultrasound probe 154, converting electrical signals corresponding to deformations in or around the boot 138 of the wireless ultrasound probe 154 into measured pressure values, processing electrical signals from the ultrasonic transducers 140 into ultrasound-image data or files, and the like. Notwithstanding the foregoing, the companion device 156 (e.g., the console 102 of FIG. 2 or the smartphone, phablet, or tablet of FIGS. 4 and 5) still includes a processor, memory, instructions, logic, etc.; however, such components need not be configured for processing electrical signals from the ultrasonic transducers 140 into ultrasound-image data or files, for example. Indeed, such components can instead be configured to display ultrasound images corresponding to the ultrasound-image data or files provided by the wireless ultrasound probe 154.

As shown, the wireless ultrasound system 152 includes a wireless ultrasound probe 154 and a companion device 156 such as the smartphone, phablet, or tablet of FIGS. 4 and 5 or, in some embodiments, the console 102 of FIG. 2. The companion device 156 includes a display 158 and a wireless module 160 configured for wireless communications with the wireless ultrasound probe 154 and, optionally, a remote Electronic Health Record (“EHR”) system. During operation of the wireless ultrasound system 152, the articulating probe head 132 of the wireless ultrasound probe 154 is placed against skin of a patient. An ultrasound beam is produced so as to ultrasonically image a portion of a target such as a blood vessel beneath a surface of the skin of the patient. The ultrasonic image of the blood vessel can be depicted on a display screen of the display 158 of the companion device 156 along with the measured pressure values by wirelessly providing data corresponding thereto from the wireless ultrasound probe 154 to the companion device 156. The wireless ultrasound system 152 is useful for assessing a target such as a blood vessel within a body of a patient before making a percutaneous puncture with a needle to place a VAD such as a catheter into the blood vessel. However, it should be appreciated that the wireless ultrasound system 152 can be useful in a variety of ultrasound-based medical procedures other than catheterization. For example, the percutaneous puncture with the needle can be performed to biopsy tissue of an organ of the patient.

FIGS. 7-11 illustrate various views of the wired ultrasound probe 106 in accordance with some embodiments; however, the wired ultrasound probe 106 and the wireless ultrasound probe 154 share at least the features as set forth below.

As shown, the wireless ultrasound probe 154 includes the probe body 130, the articulating probe head 132 attached to the probe body 130, and the pressure-sensing device 134 housed in the articulating area 136 between the articulating probe head 132 and the probe body 130. The wired ultrasound probe 106 further includes the boot 138 connecting the articulating probe head 132 to the probe body 130 in the articulating area 136. The boot 138 is configured to cover or incorporate therein the pressure-sensing device 134. The wireless ultrasound probe 154 with the articulating probe head 132 is capable of vein and catheter visualization. Like the wired ultrasound probe 106 set forth above, the wireless ultrasound probe 154 depicted in FIGS. 7-11 can be used for assessing access sites before and after placement of VADs.

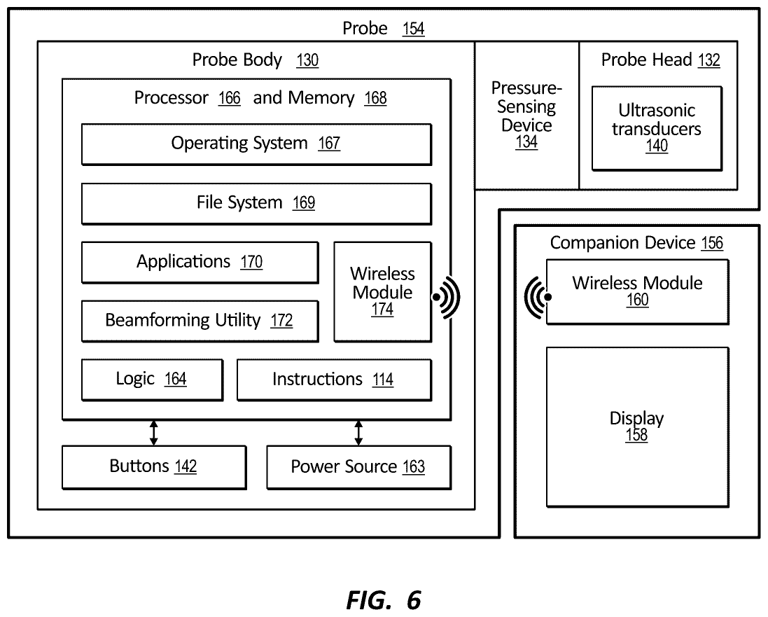

The probe body 130 houses a printed circuit board assembly (“PCBA”) 162. The PCBA 162 includes a number of electronic components of the wireless ultrasound probe 154 shown in the block diagram thereof. (See FIG. 6.) The PCBA 162 is communicatively coupled to control buttons 142 including a power button configured for toggling power to the wireless ultrasound probe 154 from power source 163 (e.g., internal battery) and various other buttons for operation of the wireless ultrasound probe 154.

Like the articulating probe head 132 of the wired ultrasound probe 106, the articulating probe head 132 of the wireless ultrasound probe 154 houses the array of ultrasonic transducers 140, wherein the ultrasonic transducers 140 are piezoelectric ultrasonic transducers or CMUTs. Again, the articulating probe head 132 is configured for placement against skin of a patient proximate a prospective VAD placement site where the ultrasonic transducers 140 in the articulating probe head 132 can generate and emit the generated ultrasound signals into the patient in a number of pulses, receive reflected ultrasound signals or ultrasound echoes from the patient by way of reflection of the generated ultrasonic pulses by the body of the patient, and convert the reflected ultrasound signals into corresponding electrical signals for processing into ultrasound images by the wireless ultrasound probe 154.

Further like the wired ultrasound probe 106, the pressure-sensing device 134 of the wireless ultrasound probe 154 is configured to detect deformations in or around the elastic material of the boot 138, which deformations are induced by pressing the articulating probe head 132 into a patient. The pressure-sensing device 134 can be a pressure transducer or a number of pressure transducers placed in the articulating area 136 between the probe body 130 and the articulating probe head 132. As set forth above, the pressure transducer can be a piezoresistive strain-gauge pressure transducer including a strain gauge bonded to a flexible diaphragm in the articulating area 136 between the articulating probe head 132 and the probe body 130. As further set forth above, the pressure transducer can be a variable capacitance pressure transducer including a diaphragm electrode and an opposing electrode in the articulating area 136 between the articulating probe head 132 and the probe body 130.

The pressure-sensing device 134 is communicatively coupled to a controller of the wireless ultrasound probe 154, which controller is optionally implemented between the processor 166 and the memory 168 of the wireless ultrasound probe 154. (See FIG. 6.) The controller is configured to convert electrical signals corresponding to the deformations in or around the elastic material of the boot 138 into the measured pressure values. The wireless ultrasound probe 154 is configured to provide the measured pressure values to the companion device 156 to be displayed to a clinician on the display screen of the companion device 156.

Notably, the wireless ultrasound probe 154 includes logic 164 configured to compare a measured pressure value against a threshold pressure value. Should the measured pressure value exceed the threshold pressure value, the wireless ultrasound probe 154 can send an electrical signal to the companion device 156 to visually or audibly alert a clinician to the foregoing measured pressure value over the threshold value. The wireless ultrasound probe 154 can additionally or alternatively include a speaker configured to emit an audio signal to alert the clinician when the measured pressure value exceeds the threshold pressure value. Additionally or alternatively, the wireless ultrasound probe 154 can include a light-emitting diode configured to emit a visual signal to alert the clinician when the measured pressure value exceeds the threshold pressure value. In this way, the wireless ultrasound probe 154 can be used to detect and determine if bodily tissue is over-compressed during ultrasound imaging. Notably, if a patient possesses excess adipose tissue, the adipose tissue can appreciably compress under the pressure induced by the articulating probe head 132. This can allow for a larger portion of, for example, a catheter to be advanced into a blood vessel. However, when the pressure induced by the articulating probe head 132 is removed, the adipose tissue can rebound causing some of the catheter to be extracted, thereby reducing the catheter purchase length. This can lead to catheter extravasation. Once the clinician is alerted of the pressure exceeding the threshold value, the clinician can check the catheter for correct placement inside the blood vessel to avoid catheter extravasation.

FIG. 6 illustrates a block diagram of the wireless ultrasound system 152 in accordance with some embodiments.

As shown, the wireless ultrasound probe 154 includes a processor 166 for governing system functionality by employment of a general-purpose operating system 167, memory 168 including a file system 169, and applications 170 that can be stored in the memory 168 and executed by the processor 166. Some of the applications 170 can provide a user interface to allow a clinician to monitor the pressure induced on a patient by the articulating probe head 132. A beamforming utility 172, including suitable circuitry is also controlled by the processor 166 to enable signals to be produced, received, and further processed. For example, the beamforming utility 172 produces electrical signals received by the ultrasonic transducers 140 in the articulating probe head 132. The articulating probe head 132 passes ultrasound signals corresponding to the electrical signals into an area of a patient and receives reflected ultrasound signals from the patient. The reflected ultrasound signals, in turn, are converted into corresponding electrical signals by the ultrasonic transducers 140 in the articulating probe head 132, which electrical signals are provided to the beamforming utility 172 for further processing into ultrasound-image data or files for display on the companion device 156. Note that the wireless ultrasound probe 154 can include different components such as more or fewer components than those set forth herein, including those components such as the wireless module 174 that enable the wireless ultrasound probe 154 to operate in a wireless manner with the companion device 156.

The wired or wireless ultrasound system 100 or 152 with the integrated pressure-sensing device 134 provides versatility beyond vein visualization for VAD placement as set forth above. Having a wired or wireless ultrasound system 100 or 152 that not only provides for ultrasound imaging but ensures that the application of the wired or wireless ultrasound probe 106 or 154 against a patient's skin does not result in excessive pressure induced by the articulating probe head 132, advantageously reduces a risk of catheter extravasation.

### Methods

Methods include a method of using the wired or wireless ultrasound system 100 or 152. For example, the method includes one or more steps selected from an ultrasound probe-obtaining step, an ultrasound probe-placing step, an ultrasound probe-moving step, a pressure-monitoring step, and a catheter placement-adjusting step.

The ultrasound probe-obtaining step includes obtaining the wired or wireless ultrasound probe 106 or 154. As set forth above, the wired and wireless ultrasound probes 106 and 154 include the probe body 130, the articulating probe head 132 attached to the probe body 130, and the pressure-sensing device 134 housed in the articulating area 136 between the articulating probe head 132 and the probe body 130.

The ultrasound probe-placing step includes placing the articulating probe head 132 of the wired or wireless ultrasound probe 106 or 154 on a skin surface of a patient.

The ultrasound probe-moving step includes moving the articulating probe head 132 of the wired or wireless ultrasound probe 106 or 154 over the patient while ultrasound signals are emitted into the patient from the articulating probe head 132 for ultrasound imaging.

The pressure-monitoring step includes monitoring for any measured pressure values induced on the patient by the articulating probe head 132 of the ultrasound probe in excess of a threshold pressure value. The monitoring can include viewing the measured pressure values on the display screen of the display 104 of the console 102 or the display 158 of the companion device 156. Such monitoring can also include monitoring for an audio signal or a visual signal on the display screen of the display 104 or 158. Such signals alert a clinician when any measured pressure values are in excess of the threshold pressure value.

The method further includes a catheter placement-adjusting step. The catheter placement-adjusting step includes adjusting catheter placement responsive to any measured pressure values in excess of the threshold pressure value to ensure a sufficient blood-vessel purchase by the catheter that minimizes catheter extravasation.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures can be made from the particular embodiments disclosed herein without departing from the scope of the concepts provided herein.

What is claimed is:

1. An ultrasound imaging method, comprising:
grasping an ultrasound probe, the ultrasound probe comprising:
an articulating probe head attached to a probe body; and
a pressure-sensing device housed in an articulating area between the articulating probe head and the probe body;
placing the articulating probe head of the ultrasound probe on a skin surface of a patient;
moving the articulating probe head of the ultrasound probe over the skin surface of the patient while ultrasound signals are emitted into the patient from the articulating probe head; and
monitoring for measured pressure values induced on the patient by the articulating probe head of the ultrasound probe to determine whether a threshold pressure value has been exceeded.

2. The ultrasound imaging method according to claim 1, further comprising viewing the measured pressure values on a display communicating with the ultrasound probe.

3. The ultrasound imaging method according to claim 2, wherein monitoring for measured pressure values includes monitoring for a visual signal on the display that indicates the threshold pressure value has been exceeded.

4. The ultrasound imaging method according to claim 1, wherein the ultrasound probe includes a speaker, and wherein monitoring for measured pressure values includes listening for an audio signal that indicates the threshold pressure value has been exceeded.

5. The ultrasound imaging method according to claim 1, wherein the ultrasound probe includes a light-emitting diode, and wherein monitoring for measured pressure values includes looking for a visual signal from the light-emitting diode that indicates the threshold pressure value has been exceeded.

6. The ultrasound imaging method according to claim 1, wherein the ultrasound probe further comprises a boot connecting the articulating probe head to the probe body in the articulating area to cover or incorporate therein the pressure-sensing device, further comprising detecting deformations in or around an elastic material of the boot induced by the articulating probe head.

7. The ultrasound imaging method according to claim 6, wherein the pressure-sensing device is communicatively coupled to a controller of the ultrasound probe, further comprising converting electrical signals corresponding to the deformations into measured pressure values by the pressure-sensing device and communicating the electrical signals to the controller.

8. The ultrasound imaging method according to claim 1, wherein the pressure-sensing device is a piezoresistive strain-gauge pressure transducer including a strain gauge bonded to a flexible diaphragm in the articulating area between the articulating probe head and the probe body, wherein a deformation in the flexible diaphragm provides a corresponding measurable change in strain-gauge electrical resistance, and wherein monitoring for measured pressure values comprises monitoring for the measurable change in strain-gauge electrical resistance.

9. The ultrasound imaging method according to claim 1, wherein the pressure-sensing device is a variable capacitance pressure transducer including a diaphragm electrode and an opposing electrode in the articulating area between the articulating probe head and the probe body, wherein a deformation in a flexible diaphragm affecting a distance between the diaphragm electrode and the opposing electrode provides a corresponding measurable change in capacitance, and wherein monitoring for measured pressure values comprises monitoring for the measurable change in the capacitance.
